(19) United States
(12) Patent Application Publication
(10) Pub. No.: US 2023/0398374 A1
(43) Pub. Date: Dec. 14, 2023

(54) MEDICAL LASER DEVICE COOPERATIVELY OUTPUTTING SOLID-STATE Q-SWITCH PULSE TM: YAG LASER AND GREEN LASER

(71) Applicants: Baogang ZHANG, Tianjin (CN); Wei SUI, Handan (CN)

(72) Inventors: Baogang ZHANG, Tianjin (CN); Wei SUI, Handan (CN)

(21) Appl. No.: 18/454,686

(22) Filed: Aug. 23, 2023

Related U.S. Application Data
(63) Continuation of application No. PCT/CN2021/105897, filed on Jul. 13, 2021.

(30) Foreign Application Priority Data
May 12, 2021 (CN) 202121004521.6

(51) Int. Cl. A61N 5/067

(52) U.S. Cl. CPC A61N 5/067; A61N 2005/0663

(57) ABSTRACT

Provided is a medical laser device cooperatively outputting solid-state Q-switch pulse TM: yAG laser and green laser, comprising a shell extending in an axial direction, and an interior of the shell is fixedly connected with a first partition plate in a radial direction; one side face of the first partition plate is fixedly connected with a second partition plate, and the interior of the shell is divided into a TM: yAG laser generating cavity, a green laser generating cavity and a light mixing cavity through the first partition plate and the second partition plate; a second totally reflective mirror, a TM: yAG rod, an acousto-optic Q-switch and a 5% spectrum output mirror are coaxially and fixedly mounted in the TM: yAG laser generating cavity, and a second 45° totally reflective mirror for reflecting laser into the light mixing cavity is fixedly mounted at a laser emitting end of the TM: yAG laser generating cavity.

### CROSS-REFERENCE TO RELATED APPLICATIONS

This application is a continuation of International Patent Application No. PCT/CN2021/105897 with a filing date of Jul. 13, 2021, designating the United States, now pending, and further claims priority to Chinese Patent Application No. 202121004521.6 with a filing date of May 12, 2021. The content of the aforementioned applications, including any intervening amendments thereto, are incorporated herein by reference.

### TECHNICAL FIELD

The application relates to the field of medical laser technologies, and is particularly a medical laser device cooperatively outputting solid-state Q-switch pulse TM: yAG laser and green laser.

### BACKGROUND OF THE PRESENT INVENTION

With the rapid development of medical science and technology, the application of laser technology in surgery is also advancing by leaps and bounds. Some scholars suggest that the future surgical technology will be separated from the era of electricity and enter the era of light. The laser technology is developing rapidly because of the following advantages of having no electrical stimulation and ensuring the safety of patients. For example, as for patients with pacemakers, an obturator nerve reflex problem in bladder surgery can be solved in the era of light; instantaneous high power makes laser become a “knife”, which can be used for cutting, coagulation, hemostasis, lithotripsy and other purposes; a heat affected zone is small and more accurate, which makes the surgery become a safe operation without accidentally injuring nerves and blood vessels of adjacent tissues; and energy is transmitted through a flexible optical fiber, and the laser can reach wherever an endoscope can reach, which makes various minimally invasive operations possible, especially to cooperate with various soft lenses in the future.

An existing TM: yAG laser emitter is in a form of single tube and single wavelength emission, and TM: yAG laser emitted by the TM: yAG laser emitter has a high water absorption rate, which is convenient for tissue gasification and is mostly used in lithotripsy. However, wavelength characteristics of the TM: yAG laser determine that the TM: yAG laser has an extremely low hemoglobin absorption rate, and cannot be used in the field of medical cosmetology.

Therefore, how to design a medical laser generator to meet a multi-functional requirement for lithotriptic cutting and medical cosmetology is a technical problem to be solved urgently by those skilled in the art.

Through public patent retrieval, the following references are found.

CN204619199U discloses a medical laser therapy apparatus, comprising a continuous laser device for emitting continuous laser; a Q-switch pulse laser device for emitting Q-switch pulse laser; and a light transmission and reflection mirror arranged on light paths of the continuous laser and the Q-switch pulse laser, so that the continuous laser and the Q-switch pulse laser are output by the light transmission and reflection mirror and then propagated along the same light path. A Gauge medical laser therapy apparatus can emit the Q-switch pulse laser and the continuous laser at the same time or separately. In one medical treatment process, when the medical laser therapy apparatus is used, the continuous laser, the Q-switch pulse laser or mixed laser of the continuous laser and the Q-switch pulse laser may be used for treatment, which is beneficial for treatment effect and treatment efficiency.

Through analysis, the laser therapy apparatus in the above patent does not disclose a laser type, a laser wavelength, and resonance generation and a transmission structure of the laser therapy apparatus, and a function and a structure of the laser therapy apparatus are quite different from those of the present application, so that the above patent does not affect the novelty of the present application.

### SUMMARY OF PRESENT INVENTION

The application aims to overcome the defects in the prior art, and provides a medical laser device cooperatively outputting solid-state Q-switch pulse TM: yAG laser and green laser, wherein green laser with a wavelength of 532 nm is complementarily matched with TM: yAG laser with a wavelength of 2,025 nm, so that a multi-functional requirement for wrinkle and epidermal erythema removal and surgical lithotripsy in medical cosmetology is met.

A medical laser device cooperatively outputting solid-state Q-switch pulse TM: yAG laser and green laser comprises a shell extending in an axial direction, wherein an interior of the shell is fixedly connected with a first partition plate in a radial direction; one side face of the first partition plate is fixedly connected with a second partition plate, and the interior of the shell is divided into a TM: yAG laser generating cavity, a green laser generating cavity and a light mixing cavity through the first partition plate and the second partition plate; a second totally reflective mirror, a TM: yAG rod, an acousto-optic Q-switch and a 5% spectrum output mirror are coaxially and fixedly mounted in sequence from a laser resonance end to a laser emitting end in the TM: yAG laser generating cavity, and a second 45° totally reflective mirror for reflecting laser into the light mixing cavity is fixedly mounted at the laser emitting end of the TM: yAG laser generating cavity; a first totally reflective mirror, a neodymium-doped yttrium aluminum garnet rod, a frequency doubling crystal and a green laser output mirror for outputting green laser into the light mixing cavity are coaxially and fixedly mounted in sequence from a laser resonance end to a laser emitting end in the green laser generating cavity; and a first 45° totally reflective mirror is adjustably connected in the light mixing cavity, and a side wall of the light mixing cavity is coupled and connected with an optical fiber for receiving laser reflected by the first 45° totally reflective mirror.

Moreover, the green laser generating cavity and the light mixing cavity are coaxially arranged, three sets of light source generators for projecting light to the neodymium-doped yttrium aluminum garnet rod are fixedly mounted on an inner wall of the green laser generating cavity, and the green laser generating cavity and the TM: yAG laser generating cavity are arranged in parallel; and three sets of palladium strips for projecting a semiconductor wave of 750 nm to 800 nm to the TM: yAG rod are fixedly mounted on an inner wall of the TM: yAG laser generating cavity.

Moreover, the light source generators and the palladium strips are all arranged in an isosceles triangle, a bottom edge of the isosceles triangle formed by the three sets of light source generators penetrates through the neodymium-doped yttrium aluminum garnet rod in a radial direction, and a bottom edge of the isosceles triangle formed by the three sets of palladium strips penetrates through the TM: yAG rod in a radial direction.

Moreover, the light source generator is semiconductor laser with a wavelength of 820 nm to 880 nm or a hernia lamp with a wavelength of 820 nm to 880 nm.

Moreover, the first partition plate and the second partition plate on a position of the light mixing cavity are both fixedly provided with a window mirror for transmitting laser.

Moreover, the second 45° totally reflective mirror outputs TM: yAG laser with a center wavelength of 2,025 nm to the first 45° totally reflective mirror through the window mirror on the first partition plate.

Moreover, the frequency doubling crystal receives green laser with a wavelength of 1,064 nm emitted by the neodymium-doped yttrium aluminum garnet rod and outputs green laser with a wavelength of 532 nm in a frequency-doubled mode; and the green laser output mirror is a lens with 532 nm wavelength output, 1,064 nm wavelength total reflection and an output rate of 1%.

Moreover, the first 45° totally reflective mirror is a three-dimensional adjustable mirror with 2,025 nm wavelength total reflection and 532 nm wavelength anti-reflection.

Moreover, the second 45° totally reflective mirror is a 2,025 nm wavelength TM: yAG laser total reflection mirror.

The application has the advantages and technical effects that:

according to the medical laser device cooperatively outputting solid-state Q-switch pulse TM: yAG laser and green laser of the application, the cooperative output of the TM: yAG laser and the green laser and the selective output of any one of the TM: yAG laser and the green laser in the interior of the same shell are realized, and the characteristics of low water absorption rate, high hemoglobin absorption rate and incapability of being used for lithotripsy of the green laser with the wavelength of 532 nm are complementarily matched with the characteristics of high water absorption rate, low hemoglobin absorption rate and good lithotripsy effect of the TM: yAG laser with the wavelength of 2,025 nm, so that a multi-functional requirement for wrinkle and epidermal erythema removal and surgical lithotripsy in medical cosmetology is met.

### DESCRIPTION OF THE DRAWINGS

FIG. 1 is a half-section view of the application; and

FIG. 2 is a side view of FIG. 1.

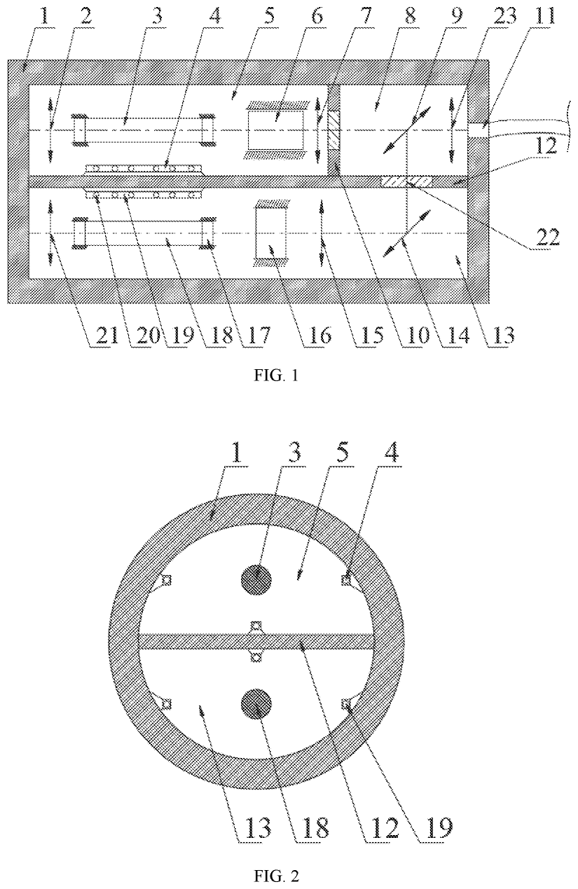

In the drawings, 1 refers to shell; 2 refers to first totally reflective mirror; 3 refers to neodymium-doped yttrium aluminum garnet rod; 4 refers to hernia lamp; 5 refers to green laser generating cavity; 7 refers to green laser output mirror; 8 refers to light mixing cavity; 9 refers to first 45° totally reflective mirror; 10 refers to second partition plate; 11 refers to optical fiber; 12 refers to first partition plate; 13 refers to TM: yAG laser generating cavity; 14 refers to second 45° totally reflective mirror; 15 refers to 5% spectrum output mirror; 16 refers to acousto-optic Q-switch; 17 refers to bonding bracket; 18 refers to TM: yAG rod; 19 refers to palladium strip; 20 refers to palladium point; 21 refers to second totally reflective mirror; 22 refers to window mirror; and 23 refers to focusing mirror.

### DETAILED DESCRIPTION OF PREFERRED EMBODIMENTS

In order to further understand the contents, characteristics and effects of the application, the following embodiments are given and explained in detail with reference to the drawings hereinafter. It should be noted that the embodiments are descriptive and nonrestrictive, and cannot limit the scope of protection of the application.

A medical laser device cooperatively outputting solid-state Q-switch pulse TM: yAG laser and green laser comprises a shell 1 extending in an axial direction, wherein an interior of the shell is fixedly connected with a first partition plate 12 in a radial direction; one side face of the first partition plate is fixedly connected with a second partition plate 10, and the interior of the shell is divided into a TM: yAG laser generating cavity 13, a green laser generating cavity 5 and a light mixing cavity 8 through the first partition plate and the second partition plate; a second totally reflective mirror 21, a TM: yAG rod 18, an acousto-optic Q-switch 16 and a 5% spectrum output mirror 15 are coaxially and fixedly mounted in sequence from a laser resonance end to a laser emitting end in the TM: yAG laser generating cavity, and a second 45° totally reflective mirror 14 for reflecting laser into the light mixing cavity is fixedly mounted at the laser emitting end of the TM: yAG laser generating cavity; a first totally reflective mirror 2, a neodymium-doped yttrium aluminum garnet rod 3, a frequency doubling crystal and a green laser output mirror 7 for outputting green laser into the light mixing cavity are coaxially and fixedly mounted in sequence from a laser resonance end to a laser emitting end in the green laser generating cavity; and a first 45° totally reflective mirror 9 is adjustably connected in the light mixing cavity, and a side wall of the light mixing cavity is coupled and connected with an optical fiber 11 for receiving laser reflected by the first 45° totally reflective mirror.

Moreover, the green laser generating cavity and the light mixing cavity are coaxially arranged, three sets of light source generators for projecting light to the neodymium-doped yttrium aluminum garnet rod are fixedly mounted on an inner wall of the green laser generating cavity, and the green laser generating cavity and the TM: yAG laser generating cavity are arranged in parallel; and three sets of palladium strips 19 for projecting a semiconductor wave of 750 nm to 800 nm to the TM: yAG rod are fixedly mounted on an inner wall of the TM: yAG laser generating cavity.

Moreover, the light source generators and the palladium strips are all arranged in an isosceles triangle, a bottom edge of the isosceles triangle formed by the three sets of light source generators penetrates through the neodymium-doped yttrium aluminum garnet rod in a radial direction, and a bottom edge of the isosceles triangle formed by the three sets of palladium strips penetrates through the TM: yAG rod in a radial direction.

Moreover, the light source generator is semiconductor laser with a wavelength of 820 nm to 880 nm or a hernia lamp 4 with a wavelength of 820 nm to 880 nm.

Moreover, the first partition plate and the second partition plate on a position of the light mixing cavity are both fixedly provided with a window mirror 22 for transmitting laser.

Moreover, the second 45° totally reflective mirror outputs TM: yAG laser with a center wavelength of 2,025 nm to the first 45° totally reflective mirror through the window mirror on the first partition plate.

Moreover, the frequency doubling crystal receives green laser with a wavelength of 1,064 nm emitted by the neodymium-doped yttrium aluminum garnet rod and outputs green laser with a wavelength of 532 nm in a frequency-doubled mode; and the green laser output mirror is a lens with 532 nm wavelength output, 1,064 nm wavelength total reflection and an output rate of 1%.

Moreover, the first 45° totally reflective mirror is a three-dimensional adjustable mirror with 2,025 nm wavelength total reflection and 532 nm wavelength anti-reflection.

Moreover, the second 45° totally reflective mirror is a 2,025 nm wavelength TM: yAG laser total reflection mirror.

In addition, according to the application, preferably, the bonding bracket is a mature product in the prior art.

In order to more clearly explain specific embodiments of the application, one embodiment is provided hereinafter.

Functions and specifications of specific parts of a medical laser device cooperatively outputting solid-state Q-switch pulse TM: yAG laser and green laser of the application are as follows.
1. A specification of a TM: yAG rod is ϕ4*102 mm, and two ends of the TM: yAG rod are respectively provided with a bonding part of 16 mm to be connected with a bonding bracket 17 for reducing a thermal lens effect, so as to facilitate heat dissipation of palladium strips.
2. The palladium strips are divided into three sets, each set of palladium strip is fixedly connected with six ammonia focused palladium points 20, and each palladium point has output power of 60 W and outputs a semiconductor wave of 750 nm to 800 nm to the palladium strip, with total power of 1,080 W.
3. An acousto-optic Q-switch is used for modulating continuous laser into a Q-switch pulse light wave, and is a mature product in the prior art.
4. A second totally reflective mirror is used for reflecting parallel light with a wavelength of 2,025 nm projected by the TM: yAG rod, the second totally reflective mirror is matched with a 5% spectrum output mirror to form a resonant cavity, and TM: yAG laser with a wavelength of 2,025 nm in the resonance cavity repeatedly passes through the TM: yAG rod and the acousto-optic Q-switch, and generates a resonant amplification effect. A specification of the second totally reflective mirror is ϕ18*3 mm, and a surface of the second totally reflective mirror is coated with a 2,025 nm wavelength totally reflective film.
5. A specification of the 5% spectrum output mirror is ϕ18*3 mm, and the 5% spectrum output mirror has a center penetrating wavelength of 2,025 nm and an output rate of 5%. The remaining 95% of the TM: yAG laser with the wavelength of 2,025 nm is reflected back to the resonant cavity for resonant amplification, which may amplify the initial output power of the TM: yAG laser with the wavelength of 2,025 nm by more than 20 times.
6. A second 45° totally reflective mirror is coated with a 2,025 nm wavelength TM: yAG laser total reflective film in center, and a specification of the second 45° totally reflective mirror is ϕ40*3 mm.
7. A specification of a window mirror is ϕ18*6 mm, and the window mirror is coated with a 2,025 nm wavelength TM: yAG laser anti-reflection film in center, which mainly plays a role of separation and sealing, and can effectively carry out light-transmitting sealing and separation on the TM: yAG laser generating cavity, the green laser generating cavity and the light mixing cavity.
8. A first 45° totally reflective mirror is coated with a 2,025 nm wavelength TM: yAG laser total reflective film and a 532 nm wavelength green laser anti-reflection film, which is a three-dimensional adjustable mirror with a specification of ϕ40*3 mm in the prior art.

A working principle of the medical laser device cooperatively outputting solid-state Q-switch pulse TM: yAG laser and green laser of the application is as follows:
the palladium strips emit semiconductor waves of 750 nm to 800 nm when energized and project the semiconductor waves to the TM: yAG rod, the TM: yAG rod is excited to generate the TM: yAG laser of 2,025 nm when the semiconductor laser reaches a certain speed, the TM: yAG laser is resonantly amplified by the resonant cavity formed by the matching between the second totally reflective mirror and the 5% spectrum output mirror, the continuous TM: yAG laser in the resonant cavity is modulated into Q-switch pulse waves with a Q-switch pulse width of 1 μs through the acousto-optic Q-switch, the output Q-switch pulse TM: yAG laser is transmitted through the second 45° totally reflective mirror and the window mirror, mixed with the green laser with the wavelength of 532 nm in the light mixing cavity, and finally reflected into the optical fiber through the first 45° totally reflective mirror for output, and it should be noted that a focusing mirror 23 in the prior art is also fixedly mounted in the light mixing cavity between the first 45° totally reflective mirror and the optical fiber.

According to the medical laser device cooperatively outputting solid-state Q-switch pulse TM: yAG laser and green laser of the application, the TM: yAG rod and the neodymium-doped yttrium aluminum garnet rod are stimulated by a side irradiation principle, with three irradiation points distributed in an isosceles triangle respectively, so to ensure stimulated areas of the TM: yAG rod and the neodymium-doped yttrium aluminum garnet rod, and the output Q-switch pulse TM: yAG laser has peak power reaching more than 30,000 watts and a Q-switch pulse width of 1 μs.

Finally, for the matters not mentioned in the application, mature products and mature technical means in the prior art are used.

It should be understood that, for those of ordinary skills in the art, improvements or transformations may be made according to the above description, and all these improvements and transformations should belong to the scope of protection of the appended claims of the application.

We claim:

1. A medical laser device cooperatively outputting solid-state Q-switch pulse TM: yAG laser and green laser, comprising a shell extending in an axial direction, wherein an interior of the shell is fixedly connected with a first partition plate in a radial direction; one side face of the first partition plate is fixedly connected with a second partition plate, and the interior of the shell is divided into a TM: yAG laser generating cavity, a green laser generating cavity and a light mixing cavity through the first partition plate and the second partition plate; a second totally reflective mirror, a TM: yAG rod, an acousto-optic Q-switch and a 5% spectrum output mirror are coaxially and fixedly mounted in sequence from a laser resonance end to a laser emitting end in the TM: yAG laser generating cavity, and a second 45° totally reflective mirror for reflecting laser into the light mixing cavity is fixedly mounted at the laser emitting end of the TM: yAG laser generating cavity; a first totally reflective mirror, a neodymium-doped yttrium aluminum garnet rod, a frequency doubling crystal and a green laser output mirror for outputting green laser into the light mixing cavity are coaxially and fixedly mounted in sequence from a laser resonance end to a laser emitting end in the green laser generating cavity; and a first 45° totally reflective mirror is adjustably connected in the light mixing cavity, and a side wall of the light mixing cavity is coupled and connected with an optical fiber for receiving laser reflected by the first 45° totally reflective mirror.

2. The medical laser device cooperatively outputting solid-state Q-switch pulse TM: yAG laser and green laser according to claim 1, wherein the green laser generating cavity and the light mixing cavity are coaxially arranged, three sets of light source generators for projecting light to the neodymium-doped yttrium aluminum garnet rod are fixedly mounted on an inner wall of the green laser generating cavity, and the green laser generating cavity and the TM: yAG laser generating cavity are arranged in parallel; and three sets of palladium strips for projecting a semiconductor wave of 750 nm to 800 nm to the TM: yAG rod are fixedly mounted on an inner wall of the TM: yAG laser generating cavity.

3. The medical laser device cooperatively outputting solid-state Q-switch pulse TM: yAG laser and green laser according to claim 2, wherein the light source generators and the palladium strips are all arranged in an isosceles triangle, a bottom edge of the isosceles triangle formed by the three sets of light source generators penetrates through the neodymium-doped yttrium aluminum garnet rod in a radial direction, and a bottom edge of the isosceles triangle formed by the three sets of palladium strips penetrates through the TM: yAG rod in a radial direction.

4. The medical laser device cooperatively outputting solid-state Q-switch pulse TM: yAG laser and green laser according to claim 2, wherein the light source generator is semiconductor laser with a wavelength of 820 nm to 880 nm or a hernia lamp with a wavelength of 820 nm to 880 nm.

5. The medical laser device cooperatively outputting solid-state Q-switch pulse TM: yAG laser and green laser according to claim 1, wherein the first partition plate and the second partition plate on a position of the light mixing cavity are both fixedly provided with a window mirror for transmitting laser.

6. The medical laser device cooperatively outputting solid-state Q-switch pulse TM: yAG laser and green laser according to claim 1, wherein the second 45° totally reflective mirror outputs TM: yAG laser with a center wavelength of 2,025 nm to the first 45° totally reflective mirror through the window mirror on the first partition plate.

7. The medical laser device cooperatively outputting solid-state Q-switch pulse TM: yAG laser and green laser according to claim 1, wherein the frequency doubling crystal receives green laser with a wavelength of 1,064 nm emitted by the neodymium-doped yttrium aluminum garnet rod and outputs green laser with a wavelength of 532 nm in a frequency-doubled mode; and the green laser output mirror is a lens with 532 nm wavelength output, 1,064 nm wavelength total reflection and an output rate of 1%.

8. The medical laser device cooperatively outputting solid-state Q-switch pulse TM: yAG laser and green laser according to claim 1, wherein the first 45° totally reflective mirror is a three-dimensional adjustable mirror with 2,025 nm wavelength total reflection and 532 nm wavelength anti-reflection.

9. The medical laser device cooperatively outputting solid-state Q-switch pulse TM: yAG laser and green laser according to claim 1, wherein the second 45° totally reflective mirror is a 2,025 nm wavelength TM: yAG laser total reflection mirror.
